(19) United States
(12) Patent Application Publication
(10) Pub. No.: US 2023/0355968 A1
(43) Pub. Date: Nov. 9, 2023

(54) ELECTROPORATION-BASED PLATFORM FOR GENERATION OF TUMOR-ACTIVATED T CELLS

(71) Applicant: Virginia Tech Intellectual Properties, Inc., Blacksburg, VA (US)

(72) Inventors: Rafael V. Davalos, Blacksburg, VA (US); Irving C. Allen, Blacksburg, VA (US); Scott S. Verbridge, Blacksburg, VA (US); Nastaran Alinezhadbalalami, Blacksburg, VA (US)

(21) Appl. No.: 18/027,824

(22) PCT Filed: Sep. 22, 2021
(86) PCT No.: PCT/US21/51551

§ 371 Date: Mar. 22, 2023

Related U.S. Application Data
(60) Provisional application No. 63/081,699, filed on Sep. 22, 2020.

(51) Int. Cl. A61N 1/32; A61N 1/36; C12N 5/0783

(52) U.S. Cl. CPC A61N 1/327; A61N 1/36002; C12N 5/0638; C12N 2529/00; C12N 2502/1121

(57) ABSTRACT

Expansion of cytotoxic T lymphocytes (CTLs) is a crucial step in almost all cancer immunotherapeutic methods. Current techniques for expansion of tumor-reactive CTLs present major limitations. The present invention comprises a novel method to effectively produce and expand tumor-activated CTLs using high-voltage pulsed electric fields. Tumor cells were subjected to high-frequency irreversible electroporation (HFIRE) with various electric field magnitudes and pulse widths, or irreversible electroporation (IRE). The treated tumor cells were subsequently cocultured with CD8+ cytotoxic T cells along with antigen-presenting cells. Tumor-activated CTLs can be produced and expanded when exposed to treated tumor cells. The activated CTLs produced with the present invention could be used for clinical applications with the goal of targeting and eliminating tumors.

### CROSS-REFERENCE TO RELATED APPLICATIONS

This application relies on the disclosure of and claims priority to and the benefit of the filing date of U.S. Provisional Application No. 63/081,699 filed Sep. 22, 2020, which is hereby incorporated by reference herein in its entirety.

### BACKGROUND OF THE INVENTION

### Field of the Invention

The present invention relates to the field of cancer immunotherapy. More specifically, embodiments of the present invention relate to using pulsed electric fields to activate and expand cytotoxic T lymphocytes, which are shown to demonstrate cytotoxicity toward tumor cells.

### Description of Related Art

Adoptive cell transfer (ACT) has become one of the major modes of cancer immunotherapy. Administration of autologous and allogeneic tumor-activated T cells is the underlying principle for the majority of fast-emerging ACT treatments. Techniques such as expansion of tumor-infiltrating lymphocytes (TILs) or engineering chimeric antigen receptor T cells (CART) are among the commonly-used methods of ACT. ACT therapies show significant promise for improving cancer treatments (Boyiadzis, M. M. et al., Chimeric antigen receptor (CAR) T therapies for the treatment of hematologic malignancies: clinical perspective and significance. J Immunother Cancer 2018, 6 (1), 137; Dafni, U. et al., Efficacy of adoptive therapy with tumor-infiltrating lymphocytes and recombinant interleukin-2 in advanced cutaneous melanoma: a systematic review and meta-analysis. Ann Oncol 2019, 30 (12), 1902-1913). Despite the encouraging early results, several limitations persist such as low initial TIL numbers, the time-consuming process of expansion, and identifying reliable targets for CAR T cells.

ACT therapies using TILs suffer from limited numbers of tumor-infiltrating lymphocytes. TILs can only be harvested from tumor tissue and are usually low in quantity, especially in immunologically “cold” tumors (Hall, M. et al., Expansion of tumor-infiltrating lymphocytes (TIL) from human pancreatic tumors. J Immunother Cancer 2016, 4, 61; Paijens, S. T. et al., Tumor-infiltrating lymphocytes in the immunotherapy era. Cellular & Molecular Immunology 2020). CAR T cell therapies can avoid this limitation by employing lymphocytes from peripheral blood that are easier to obtain. However, recognition of tumor-specific antigens for designing effective, on-target CARs can be challenging due to heterogeneity of antigen expression in tumors. Furthermore, the efficiency of CAR T cell therapies is limited due to on-target off-tumor toxicity which can result in severe side effects (Kailayangiri, S. et al., Overcoming Heterogeneity of Antigen Expression for Effective CART Cell Targeting of Cancers. Cancers (Basel) 2020, 12 (5); Richman, S. A. et al., High-Affinity GD2-Specific CAR T Cells Induce Fatal Encephalitis in a Preclinical Neuroblastoma Model. Cancer Immunol Res 2018, 6 (1), 36-46; Morgan, R. A. et al., Case report of a serious adverse event following the administration of T cells transduced with a chimeric antigen receptor recognizing ERBB2. Mol Ther 2010, 18 (4), 843-51). Additionally, both CAR T cells and TILs are often expanded through stimulation with IL-2 (interleukin 2), sometimes in combination with antibodies such as anti-CD28 for over 2-3 weeks (Turcotte, S. et al., Phenotype and function of T cells infiltrating visceral metastases from gastrointestinal cancers and melanoma: implications for adoptive cell transfer therapy. J Immunol 2013, 191 (5), 2217-25; Riddell, S. R. et al., The use of anti-CD3 and anti-CD28 monoclonal antibodies to clone and expand human antigen-specific T cells. journal of immunological methods 1990, 128 (2), 189-201). As a result, the expansion of activated T cells is expensive and time consuming.

Several platforms have been developed with the goal of producing patient-derived tumor-reactive T cells (Hall, 2016; Dijkstra, K. K. et al., Generation of Tumor-Reactive T Cells by Co-culture of Peripheral Blood Lymphocytes and Tumor Organoids. Cell 2018, 174 (6), 1586-1598 e12; Poch, M. et al., Expansion of tumor infiltrating lymphocytes (TIL) from bladder cancer, Oncoimmunology 2018, 7 (9), e1476816) by expanding either TILs or CTLs obtained from the peripheral blood. Dijkstra et al. proposed production of tumor-reactive CTLs by exposing autologous peripheral blood lymphocytes to colorectal tumor organoids. The organoids were stimulated with IFN-γ prior to coculture with lymphocytes. Furthermore, the proliferation of T cells was supported by culturing them in plate-bound CD28 antibodies along with stimulation with IL-2 (Dijkstra, 2018). In a similar manner, solid tumor biopsies were used for the expansion of tumor-infiltrating lymphocytes (TILs), stimulating them with IL-2 and IFN-γ (Hall, 2016; Poch, 2018). A majority of prior studies used cytokines or antibodies to expand CTLs or TILs.

Other studies include Zhao, J. et al., Irreversible electroporation reverses resistance to immune checkpoint blockade in pancreatic cancer. Nat Commun 2019, 10 (1), 899; Shao, Q. et al., Engineering T cell response to cancer antigens by choice of focal therapeutic conditions. Int J Hyperthermia 2019, 36 (1), 130-138. As provided in embodiments of the present invention, however, the inventors are the first to introduce a coculture method for production of human CTLs using HFIRE.

Some key differences are expected between IRE and HFIRE cell death due to the difference in applied pulse scheme. It has been shown that the IRE mostly induces apoptosis, pyroptosis, necroptosis and/or necrosis. An additional delayed inflammatory cell death is identified for HFIRE treatments, closer to pyroptosis (Brock, R. M. et al., Starting a Fire Without Flame: The Induction of Cell Death and Inflammation in Electroporation-Based Tumor Ablation Strategies. frontiers in oncology 2020, 10; Ringel-Scaia, V. M. et al., High-frequency irreversible electroporation is an effective tumor ablation strategy that induces immunologic cell death and promotes systemic anti-tumor immunity. EBioMedicine 2019, 44, 112-125; Mercadal, B. et al., Dynamics of Cell Death After Conventional IRE and H-FIRE Treatments. Ann Biomed Eng 2020, 48 (5), 1451-1462; Batista Napotnik, T. et al., Cell death due to electroporation—A review. Bioelectrochemistry 2021, 141, 107871). It is important to note that the cell death mechanism can also vary based on the applied electric field and the pulse duration. It is known that the pulse durations that are well below the membrane charging time, would lead to different pore dynamics on the cell membrane as well as potential effects on the intercellular organelles (Weaver, J. C. et al., A brief overview of electroporation pulse strength-duration space: a region where additional intracellular effects are expected. Bioelectrochemistry 2012, 87, 236-43; Sano, M. B. et al., In-vitro bipolar nano- and microsecond electro-pulse bursts for irreversible electroporation therapies. Bioelectrochemistry 2014, 100, 69-79; Schoenbach, K. H. et al., Intracellular effect of ultrashort electrical pulses. Bioelectromagnetics 2001, 22 (6), 440-8). Even though these differences in pore dynamics would change if pulse width increases, such changes are expected to be gradual (Weaver, 2012). Hence, some differences among the HIRE treatment groups could be imagined. The scope of antigen presentation and T cell activation can differ based on the cell death mechanism resulted from the IRE or HFIRE treatments.

### SUMMARY OF THE INVENTION

The present inventors have developed methods of using pulsed electric fields to activate and/or expand cytotoxic T lymphocytes (CTLs). According to embodiments, tumor-reactive T cells and/or cytokines can be activated by subjecting cells to a plurality of electrical pulses, such as HFIRE, and exposing the cells to helper T cells and/or cytotoxic T cells. The activated tumor-reactive T cells and/or cytokines can then be used to stimulate the immune system of a subject to act against tumor cells and/or further stimulate/activate CTLs of the subject.

Embodiments include any of the following Aspects, such as Aspect 1, which is a method comprising: subjecting one or more patient cell (e.g., tumor cells) to a plurality of electrical pulses, wherein the subjecting is performed in vitro or in vivo or ex vivo; optionally exposing one or more of the patient cells to peripheral blood mononuclear cells (PBMCs), tumor infiltrating lymphocytes (TILs), or dendritic cells; optionally exposing one or more of the patient cells to CD4+ helper T cells and/or CD8+ cytotoxic T cells (CTLs); generating, activating and/or providing for expansion of one or more tumor-reactive T cells, such as CD4+ cells and/or CD8+ cells, and/or cytokines, such as IL-2 and/or IFN-γ; optionally separating activated cells (such as CD4+ and/or CD8+) and/or cytokines out; optionally inoculating or exposing a patient with/to the activated tumor-reactive T cells and/or activated cytokines, for example, to trigger an immune response in the patient, such as against one or more tumor, and/or further stimulation of CTLs in the patient.
Aspect 2 is a method for producing cytotoxic T lymphocytes, comprising: positioning one or more electrodes near a target area containing cells to be treated; applying a plurality of electrical pulses to the target area through the electrodes; exposing treated cells to dendritic cells; and exposing the treated cells to one or more types of T lymphocytes.
Aspect 3 is the method of Aspect 1 or 2, wherein the plurality of electrical pulses constitutes high frequency irreversible electroporation (HFIRE).
Aspect 4 is the method of any of Aspects 1-3, wherein no cytokines or antibodies are added.
Aspect 5 is the method of any of Aspects 1-4, wherein the plurality of electrical pulses are administered at a voltage in the range of above 0 V to 10,000 V.
Aspect 6 is the method of any of Aspects 1-5, wherein pulses of the plurality of electrical pulses have a pulse width in the range of from about 1 picosecond to 50 microseconds.
Aspect 7 is the method of any of Aspects 1-6, wherein the plurality of electrical pulses comprise HFIRE pulses administered according to a positive pulse-delay-negative pulse protocol or a negative pulse-delay-positive pulse protocol.
Aspect 8 is the method of any of Aspects 1-7, wherein the plurality of electrical pulses has a frequency in the range of above 0 Hz to 100 MHz.
Aspect 9 is the method of any of Aspects 1-8, wherein the plurality of electrical pulses comprise HFIRE pulses of alternating polarity administered according to a pulse protocol having a format of ontime-offtime-ontime.
Aspect 10 is the method of any of Aspects 1-9, wherein one or more pulses of the plurality of electrical pulses are monopolar or bipolar.
Aspect 11 is the method of any of Aspects 1-10, wherein one or more pulses of the plurality of electrical pulses has a square waveform.
Aspect 12 is the method of any of Aspects 1-11, wherein the plurality of electrical pulses has a pulsing scheme with one or more intra- or inter-pulse delays.
Aspect 13 is the method of any of Aspects 1-12, wherein the plurality of electrical pulses has a pulsing scheme conforming to the following formula: (i) administering a pulse with a first polarity and a pulse duration of less than 10 microseconds, (ii) administering a delay with a duration of up to 20 microseconds, (iii) administering a pulse with a second polarity, which is the same or a different as the first polarity, and a pulse duration of less than 10 microseconds, (iv) administering a delay of up to 1 second, and (v) repeating the administering of (i)-(iv) a desired number of times.
Aspect 14 is the method of any of Aspects 1-13, wherein any number of pulses is administered, wherein there are a total number of pulses, and/or a total number of pulses per burst, ranging from 1-5,000 pulses.
Aspect 15 is a method comprising: applying a plurality of electrical pulses to a cell sample in vitro or ex vivo; culturing the cell sample with human dendritic cells; exposing the cell sample and human dendritic cells to human helper T cells and/or cytotoxic T cells, such that the cytotoxic T cells proliferate; and isolating the cytotoxic T cells from the cell sample.
Aspect 16 is the method of any of Aspects 1-15, wherein the cells or cell sample comprise tumor cells.
Aspect 17 is the method of any of Aspects 1-16, wherein the human helper T cells are CD4+ helper T cells and/or the cytotoxic T cells are CD8+ cytotoxic T cells.
Aspect 18 is the method of any of Aspects 1-17, wherein the exposing involves using human CD4+ helper T cells and/or CD8+ cytotoxic T cells which are isolated from peripheral blood mononuclear cells or are isolated from an organ, such as the spleen.
Aspect 19 is the method of any of Aspects 1-18, further comprising administering the isolated CD8+ cytotoxic T cells to a patient.
Aspect 20 is the method of any of Aspects 1-19, further comprising exposing the isolated CD8+ cytotoxic T cells to other cells or a second cell sample in vitro or ex vivo, wherein the other cells or second cell sample are from an individual who is the same or different as from which the first cells or cell sample were obtained.
Aspect 21 is the method of any of any of Aspects 1-20, wherein the plurality of electrical pulses are bipolar pulses and/or comprise a square waveform and/or comprise one or more intra-pulse or inter-pulse delay.
Aspect 22 is the method of any of Aspects 1-21, wherein the plurality of electrical pulses have a square waveform and/or pulses of the plurality of electrical pulses are asymmetric.
Aspect 23 is the method of any of Aspects 1-22, further comprising exposing the cells or cell sample and human dendritic cells to cytokines and/or antibodies.
Aspect 24 is the method of any of Aspects 1-23, wherein during the applying, the cell sample is a suspension.
Aspect 25 is a method comprising: obtaining and isolating a cell sample from a first subject; applying a plurality of electrical pulses to one or more cells of the cell sample; culturing the one or more cells with dendritic cells; exposing the one or more cells and dendritic cells to helper T cells and/or cytotoxic T cells, such that the activated T cells proliferate, such as the cytotoxic T cells proliferate; and isolating the cytotoxic T cells.
Aspect 26 is the method of any of Aspects 1-25, further comprising administering the isolated cytotoxic T cells into a subject, such as the first subject.
Aspect 27 is the method of any of Aspects 1-26, further comprising administering the isolated cytotoxic T cells into a second cell sample or into a second subject, which second cell sample or second subject is the same or a different cell sample or subject from the first subject.
Aspect 28 is the method of any of Aspects 1-27, wherein the cell sample comprises tumor cells or is a tumor biopsy.
Aspect 29 is the method of any of Aspects 1-28, further comprising suspending the cells or cell sample in a liquid medium prior to applying the plurality of electrical pulses.
Aspect 30 is the method of any of Aspects 1-29, wherein the plurality of electrical pulses are bipolar pulses.
Aspect 31 is the method of any of Aspects 1-30, wherein one or more of the plurality of electrical pulses are bipolar pulses.
Aspect 32 is the method of any of Aspects 1-31, further comprising exposing the one or more cells, or the one or more cells and human dendritic cells, to cytokines and/or antibodies.
Aspect 33 is a method comprising: applying a plurality of electrical pulses to one or more cells of a subject in vivo; isolating one or more of the cells of the subject; culturing the one or more cells with dendritic cells; exposing the one or more cells and dendritic cells to helper T cells and/or cytotoxic T cells; and isolating the cytotoxic T cells.
Aspect 34 is the method of any of Aspects 1-33, further comprising administering the isolated cytotoxic T cells to the subject or to a second subject, optionally administering additional HFIRE or IRE to the subject or to the second subject.
Aspect 35 is a method comprising: applying a plurality of electrical pulses to a cell sample in vitro or ex vivo; and culturing the cell sample with human dendritic cells.
Aspect 36 is the method of any of Aspects 1-35, further comprising exposing the cell sample and human dendritic cells to human helper T cells and/or cytotoxic T cells, optionally further administering HFIRE or IRE to the cells in vitro or ex vivo after such exposing.
Aspect 37 is the method of any of Aspects 1-36, further comprising isolating the cytotoxic T cells from the cell sample.
Aspect 38 is the method of any of Aspects 1-37, wherein the cell sample and human dendritic cells are exposed to the human helper T cells and/or cytotoxic T cells for at least 12 hours.
Aspect 39 is the method of any of Aspects 1-38, wherein the cell sample and human dendritic cells are exposed to the human helper T cells and/or cytotoxic T cells for up to 96 hours.
Aspect 40 is the method of any of Aspects 1-39, wherein the cytotoxic T cells are isolated from the cell sample by way of magnetic-activated cell sorting.
Aspect 41 is the method of any of Aspects 1-40, wherein the plurality of electrical pulses are administered at a voltage in the range of from 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or for example from 100 V to 15,000 V, such as from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 15,000 V, up to 7,500 V, from 4,000 V to 12,000 V, such as less than 450 V, or less than 425 V, such as from above 0 V to 400 V, or from about 10 V to 350 V, or about 20 V to about 300 V, or about 30 V to about 250 V, or from about 15 V to about 200 V, or from about 50 V to about 150 V, or about 75 V to 100 V, or from 30 V to 225 V, or from 60 V to 375 V.
Aspect 42 is the method of any of Aspects 1-41, wherein the pulses have a pulse width in the range of from about 10 ns to about 200 microseconds, or from about 10 ns to about 10 microseconds, or about 10 microseconds or less, or any range in between any of these ranges or endpoints, including as endpoints any number encompassed thereby.
Aspect 43 is the method of any of Aspects 1-42, wherein one or more pulses of the plurality of electrical pulses has a pulse length in the picosecond to microsecond range, such as in the nanosecond to microsecond range, including from 1 picosecond to below 10 microseconds, or from 1 picosecond to 1 microsecond, or below 1 microsecond, or from at least 0.1 microsecond up to 5 microseconds, or from 0.5 microseconds up to 2 microseconds or up to 10 microseconds, or any range in between any of these ranges or endpoints, including as endpoints any number encompassed thereby, such as a high-frequency irreversible electroporation burst scheme of pulse width and intra-phase delay ranging from 0.1 μs to 10 ms and an inter-pulse delay ranging from 0.1 μs to 1 s.
Aspect 44 is the method of any of Aspects 1-43, wherein the pulse and/or burst repetition rate or frequency is from 1 Hz up to 100 MHz, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or a frequency ranging from 100 Hz to 100 MHz, such as in the Hz range from 1 Hz up to 100 Hz, or from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency in the kHz or MHz range, such as from 1 kHz to 10 kHz, or from 2 kHz to 8 kHz, or from 3 kHz to 5 kHz, or from 4 kHz to 9 kHz, or from 7 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 10-25 kHz, or from 15-40 kHz, or from 20-50 kHz, or from 75 kHz to 150 kHz, or from 100 kHz to 175 kHz, or from 200 kHz to 250 kHz, or from 225 kHz to 500 kHz, or from 250 kHz to 750 kHz, or from 500 kHz to 1 MHz, or any range in between any of these ranges or endpoints, including as endpoints any number encompassed thereby.
Aspect 45 is the method of any of Aspects 1-44, wherein the plurality of electrical pulses comprises pulses with a waveform comprising alternating polarity.
Aspect 46 is the method of any of Aspects 1-45, wherein one or more pulses of the plurality of electrical pulses has a triangular, trapezoidal, exponential decay, sawtooth, sinusoidal, and/or such waveforms comprising one or more pulses of alternating polarity.
Aspect 47 is the method of any of Aspects 1-46, wherein the pulsing scheme comprises a pulse length-delay-pulse length pulsing scheme chosen from: 1-1-1 μs, 2-1-2 μs, 5-1-5 μs, or 10-1-10 μs, with up to a 1-second delay between bursts.
Aspect 48 is the method of any of Aspects 1-47, wherein any number of pulses is administered.
Aspect 49 is the method of any of Aspects 1-48, wherein there are a total number of pulses, and/or a total number of pulses per burst, ranging from 1-5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or the total number of pulses and/or bursts ranges from 1 to 5,000 pulses/bursts, such as from at least 1 up to 3,000 pulses/bursts, or at least 2 up to 2,000 pulses/bursts, or at least 5 up to 1,000 pulses and/or bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIGS. 1A-G are illustrations showing steps of a T cell expansion platform according to an embodiment of the invention: (A) tumor cell isolation; (B) HFIRE treatment; (C) cell culture; (D) tissue mimic; (E) T cell isolation; (F) applications; (G) cell type key.

FIGS. 2A-D are graphs showing HFIRE and IRE pulse schemes according to embodiments of the invention: (A) HFIRE 1-1-1 bipolar pulses; (B) HFIRE 5-1-5 bipolar pulses; (C) HFIRE 10-1-10 bipolar pulses; (D) IRE monopolar pulses.

FIG. 3AB are graphs showing the cell viability (3A) and temperature rise (3B) after HFIRE and IRE treatment.

FIG. 4A is an illustration showing the dilution of the cytoplasmic dye concentration with each cell division.

FIG. 4B is a graph showing various fluorescent intensity peaks corresponding to each generation of proliferative ells.

FIGS. 4C-G are graphs showing the results of CFSE analysis for cells treated with each electroporation method: (C) HFIRE 1-1-1, 5-1-5, and 10-1-10 at 1250V/cm; (D) HFIRE 1-1-1, 5-1-5, and 10-1-10 at 2500V/cm; (E) no treatment; (F) IRE; (G) mechanical lysis.

FIGS. 5A-B are graphs showing the proliferation index (5A) and percent division (5B) for all treatment groups.

FIGS. 6A-B are graphs showing ELISA results for IL-2 levels (6A) and IFN-γ levels (6B) in the coculture system after HFIRE and IRE stimulation.

FIGS. 7A-C are illustrations showing the treatment of tumor cells with HFIRE (7A), CD8 T cell isolation (7B), and addition of T cells to untreated U251 cells (7C).

FIG. 7D is a graph showing the percent toxicity of the CTLs as determined through an LDH assay.

FIG. 8 is an illustration showing an exemplary process of T cell activation after administration of electroporation treatment.

### DETAILED DESCRIPTION OF VARIOUS EMBODIMENTS OF THE INVENTION

Recent in vivo studies using electroporation-based cancer treatments suggest that in addition to ablation of tumors, electroporation treatments can stimulate an immune response (Neal, R. E., 2^{nd} et al., Improved local and systemic anti-tumor efficacy for irreversible electroporation in immunocompetent versus immunodeficient mice. PLoS One 2013, 8 (5), e64559; Partridge, B. R. et al., High-Frequency Irreversible Electroporation for Treatment of Primary Liver Cancer: A Proof-of-Principle Study in Canine Hepatocellular Carcinoma. J Vasc Intery Radiol 2020, 31 (3), 482-491 e4; Falk, H. et al., Electrochemotherapy and calcium electroporation inducing a systemic immune response with local and distant remission of tumors in a patient with malignant melanoma—a case report. Acta Oncol 2017, 56 (8), 1126-1131). Electroporation therapies, such as electrochemotherapy (ECT) (Mali, B. et al., Antitumor effectiveness of electrochemotherapy: a systematic review and meta-analysis. Eur J Surg Oncol 2013, 39 (1), 4-16; Gothelf, A. et al., Electrochemotherapy: results of cancer treatment using enhanced delivery of bleomycin by electroporation. Cancer Treatment Reviews 2003, 29 (5), 371-387), irreversible electroporation (IRE) (Davalos, R. V. et al., Tissue ablation with irreversible electroporation. Ann Biomed Eng 2005, 33 (2), 223-31; Scheffer, H. J. et al., Irreversible electroporation for nonthermal tumor ablation in the clinical setting: a systematic review of safety and efficacy. J Vasc Intery Radiol 2014, 25 (7), 997-1011; quiz 1011; Golberg, A. et al., Nonthermal irreversible electroporation: fundamentals, applications, and challenges. IEEE Trans Biomed Eng 2013, 60 (3), 707-14; Geboers, B. et al., High-Voltage Electrical Pulses in Oncology: Irreversible Electroporation, Electrochemotherapy, Gene Electrotransfer, Electrofusion, and Electroimmunotherapy. Radiology 2020, 295 (2), 254-272), and high-frequency irreversible electroporation (HFIRE) (Arena, C. B. et al., High-frequency irreversible electroporation (H-FIRE) for non-thermal ablation without muscle contraction. Biomedical engineering online 2011, 10 (1), 102; Wasson, E. M. et al., Understanding the role of calcium-mediated cell death in high-frequency irreversible electroporation. Bioelectrochemistry 2020, 131, 107369) use high-voltage electrical pulses to destabilize the cell membrane by raising the transmembrane potential (TMP). The elevation of TMP beyond a threshold leads to formation of defects (pores) in the cell membrane and enables diffusion of water and other molecules such as therapeutic reagents into the cytoplasm (Kotnik, T. et al., Membrane Electroporation and Electropermeabilization: Mechanisms and Models. Annu Rev Biophys 2019, 48, 63-91). Membrane disruption can be reversible; however, if sufficient energy (voltage and duration) is applied, cell death will occur. ECT exploits reversible membrane permeabilization that enhances chemotherapy uptake by cells and leads to their eventual death. IRE and HFIRE treatments, however, employ electric fields above the lethal threshold, causing irreversible membrane destabilization, loss of homeostasis, and cell death. As a result, the cell death induced by IRE and HFIRE is mainly dependent on the nature of the applied electric pulses.

Among electroporation techniques, IRE is the most commonly used tissue ablation technique in the clinic (Aycock, K. N., Rafael V. Davalos, Irreversible Electroporation: Background, Theory, and Review of Recent Developments in Clinical Oncology. Bioelectricity 2019, 1 (4), 214-234). IRE is a non-thermal, minimally invasive treatment modality in which multiple (such as 80-100) monopolar pulses, often with 100 μs pulse duration, are administered through a number of electrodes. HFIRE is the modified technology that was previously invented with the goal of reducing undesirable IRE side effects (Arena, 2011). In HFIRE treatments, the voltage is applied in the format of bursts that consist of bipolar pulses (often on the order of 0.5-10 μs) with an inter-pulse delay. Similar to IRE, 80-100 bursts are often delivered with a total on-time of around 100 μs for each burst. Delivering shorter pulses with high-frequencies may have unique advantages, such as organelle damage due to penetration of the field into the intracellular space and a different cell death mechanism comparing to longer pulses (Weaver, 2012; Sano, 2014; Schoenbach, 2001).

Despite their differences, immunological cell death can arise from both IRE and HFIRE treatments (Wasson, 2020; Ringel-Scaia, V. M. et al., High-frequency irreversible electroporation is an effective tumor ablation strategy that induces immunologic cell death and promotes systemic anti-tumor immunity. EbioMedicine 2019, 44, 112-125; Goswami, I. et al., Irreversible electroporation inhibits pro-cancer inflammatory signaling in triple negative breast cancer cells. Bioelectrochemistry 2017, 113, 42-50). Recently, significant effort has been focused on either characterizing or deploying the immune response resulting from inflammatory cell death following pulsed electric field treatments. A tumor-suppressive immune response after IRE and HFIRE treatments has also been reported in several clinical studies and animal models both after IRE (Neal, 2013; Zhao, 2019; Narayanan, J. S. S. et al., Irreversible Electroporation Combined with Checkpoint Blockade and TLR7 Stimulation Induces Antitumor Immunity in a Murine Pancreatic Cancer Model. Cancer Immunol Res 2019, 7 (10), 1714-1726; He, C. et al., Immunomodulatory Effect after Irreversible Electroporation in Patients with Locally Advanced Pancreatic Cancer. J Oncol 2019, 2019, 9346017; Go, E. J. et al., Combination of Irreversible Electroporation and STING Agonist for Effective Cancer Immunotherapy. Cancers (Basel) 2020, 12 (11); Beitel-White, N. et al., Real-time prediction of patient immune cell modulation during irreversible electroporation therapy. Sci Rep 2019, 9 (1), 17739) and HFIRE pulses (Partridge, 2020; Ringel-Scaia, 2019). Furthermore, various studies show increased effectiveness of IRE treatments when combined with recently developed immunotherapy methods such as blockade of checkpoint inhibitors (Zhao, 2019; Narayanan, 2019; Go, 2020). The combination treatment of IRE with anti-PD1 (programmed cell death protein 1) was shown to be effective in triggering and sustaining an immune response that leads to tumor suppression and increased survival. The systemic immune response is suggested to be due to the upregulation of cytotoxic T cell activity (Zhao, 2019; Nuccitelli, R. et al., Nanoelectroablation of Murine Tumors Triggers a CD8-Dependent Inhibition of Secondary Tumor Growth. PLoS One 2015, 10 (7), e0134364).

The overall mechanism of cell death and its immune-stimulatory effects are less explored for HFIRE treatments. However, there is evidence for T cell-dependent immune response after HFIRE treatments (Partridge, 2020). In two recent studies, an alteration in gene expression patterns was observed after HFIRE treatments that led to pro-inflammatory cell death in brain and breast tumor cells (Wasson, 2020; Ringel-Scaia, 2019). Given the stimulatory nature of HFIRE in vivo, tumor-reactive T cells could be generated and expanded in vitro or ex vivo with exposure of T cells to HFIRE treated tumor cells. Furthermore, HFIRE-induced activated CTLs are expected to show cytotoxicity against untreated tumor cells. It has been suggested that the non-thermal nature of electroporation-based cell death allows for preserving proteins during treatment, likely leading to more effective antigen presentation compared to other techniques such as cyro- and thermal ablation (Brock, 2020).

According to an embodiment of the present invention, cancer cells treated with electroporation are cocultured with dendritic cells (DCs) leading to DC maturation and antigen presentation. CD4+ and CD8+ T cells are added to the coculture system, resulting in activation and proliferation of CTLs. DCs are the most potent antigen presenting cells and are capable of presenting a variety of antigens and triggering a CTL-based systemic immune response. In the coculture system, the antigens are presented by DCs through class I and class II major histocompatibility complex (MHC) molecules upon release from the treated tumor cells. Direct activation of CD8+ T cells can occur through MHC I. MHC II interacts with TCRs (T cell receptors) on CD4+ T cells along with co-stimulation with CD28 and B7. The activated CD4+ helper cells contribute to the activation of CD8+ T cells through cytokine secretion such as IL-2. Hence, both CD4+ and dendritic cells play an active role in generation of tumor-activated CD8+ T cells.

Various HFIRE pulse widths at two different electric field strengths are included in the following example. An IRE treatment group is included for comparison. In addition, the T cell response of HFIRE treatment is compared to mechanical disruption of the cell membrane.

In embodiments, the present invention does not require addition of any cytokines or antibodies for production of tumor-reactive lymphocytes. Furthermore, the coculture system can be utilized to understand the dynamics of HFIRE-induced T cell activation and antigen presentation. As ACT therapies continue to be important in immune-oncology, HFIRE may be a beneficial alternative for expanding and activating CTLs ex vivo.

In embodiments of the invention, electroporation is administered by way of a plurality of electrical pulses, wherein one or more pulses of the plurality of electrical pulses has a pulse length in the picosecond to microsecond range, such as in the nanosecond to microsecond range, including from 1 picosecond to below 10 microseconds, or from 1 picosecond to 1 microsecond, or below 1 microsecond, or from at least 0.1 microsecond up to 5 microseconds, or from 0.5 microseconds up to 2 microseconds or up to 10 microseconds, such as up to 100 ns, 250 ns, 500 ns, 1 μs, 2 μs, 5 μs, 10 μs, 15 μs, 20 μs, 25 μs, 40 μs, 50 μs, 75 μs, 100 μs, 125 μs, or 150 μs, or even up to about 200 μs or any range in between any of these ranges or endpoints, including as endpoints any number encompassed thereby, such as a high-frequency irreversible electroporation burst scheme of pulse width and intra-phase delay ranging from 0.1 μs to 10 ms and an inter-pulse delay ranging from 0.1 μs to 1 s.

In embodiments, the plurality of electrical pulses can have a pulsing scheme that incorporates one or more intra- or inter-pulse delays and/or one or more intra- or inter-burst delays, such as pulsing schemes of bursts of pulses comprising schemes of 1-1-1 μs, 2-1-2 μs, 5-1-5 μs, or 10-1-10 μs with up to a 1-second delay between bursts.

In embodiments, the plurality of electrical pulses may be administered with no delay, or effectively no delay, between pulses or with a delay between pulses and/or between bursts of pulses. If administered with an inter-pulse delay, the delay between pulses can be up to 100 times, 50 times, 20 times, 10 times, or 5 times the pulse length, such as 3 times the pulse length, 2 times the pulse length, equal to the pulse length, or less than the pulse length. For example, the delay between pulses can be 10%, 20%, 25%, 40%, 50%, 60%, 75%, 80%, or 90% of the pulse length.

In embodiments, the plurality of electrical pulses administered can be monopolar or bipolar pulses. If bipolar pulses are administered, the pulses can change polarity instantly or with an intra-pulse delay. For example, the intra-pulse delay can be up to 5 times the pulse length, such as 3 times the pulse length, 2 times the pulse length, equal to the pulse length, or less than the pulse length. For example, the intra-pulse delay can be 1%, 5%, 10%, 20%, 25%, 40%, 50%, 60%, 75%, 80%, or 90% of the pulse length. For bipolar pulses, the positive and negative applied voltages do not have to be of equal magnitude.

In embodiments of the invention, the plurality of electrical pulses are administered at a voltage in the range of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or for example from 100 V to 15,000 V, such as from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 15,000 V, up to 7,500 V, from 4,000 V to 12,000 V, such as less than 450 V, or less than 425 V, such as from above 0 V to 400 V, or from about 10 V to 350 V, or about 20 V to about 300 V, or about 30 V to about 250 V, or from about 15 V to about 200 V, or from about 50 V to about 150 V, or about 75 V to 100 V, or from 30 V to 225 V, or from 60 V to 375 V.

In embodiments, the number of pulses is administered and/or a total number of pulses per burst, ranges from 1-5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or the total number of pulses and/or bursts can range from 1 to 5,000 pulses/bursts, such as from at least 1 up to 3,000 pulses/bursts, or at least 2 up to 2,000 pulses/bursts, or at least 5 up to 1,000 pulses and/or bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby.

In embodiments, the pulse rate can have a frequency in the range of about 0 Hz to 100 MHz, such as from above 0 Hz or 1 Hz up to 100 MHz, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or a frequency ranging from 100 Hz to 100 MHz, such as in the Hz range from 100 Hz or 1 Hz up to 100 Hz, or from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency in the kHz or MHz range, such as from 1 kHz to 10 kHz, or from 2 kHz to 8 kHz, or from 3 kHz to 5 kHz, or from 4 kHz 9 kHz, or from 7 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 10-25 kHz, or from 15-40 kHz, or from 20-50 kHz, or from 75 kHz to 150 kHz, or from 100 kHz to 175 kHz, or from 200 kHz to 250 kHz, or from 225 kHz to 500 kHz, or from 250 kHz to 750 kHz, or from 500 kHz to 1 MHz, or any range in between any of these ranges or endpoints, including as endpoints any number encompassed thereby.

In embodiments, the shape of the electrical pulses delivered can be any desired waveform, including square, triangular, trapezoidal, exponential decay, sawtooth, sinusoidal, and/or such waveforms comprising one or more pulses of alternating polarity.

### EXAMPLE

Embodiments of the present invention include a coculture method that uses electroporation-based therapies to activate and expand primary human CTLs against tumors. In the coculture system, primary human dendritic cells are exposed to tumor cells previously treated with IRE or HFIRE pulses. The release of antigens from treated tumor cells leads to the maturation of the dendritic cells. Upon activation of DCs, human CD4+ helper T cells and/or CD8+ cytotoxic T cells, isolated through negative selection from peripheral blood mononuclear cells (PBMCs) or isolated from an organ, such as the spleen, are added to the coculture system. Antigen presentation through class I and class II MHC molecules on dendritic cells leads to direct or indirect activation of CTLs.

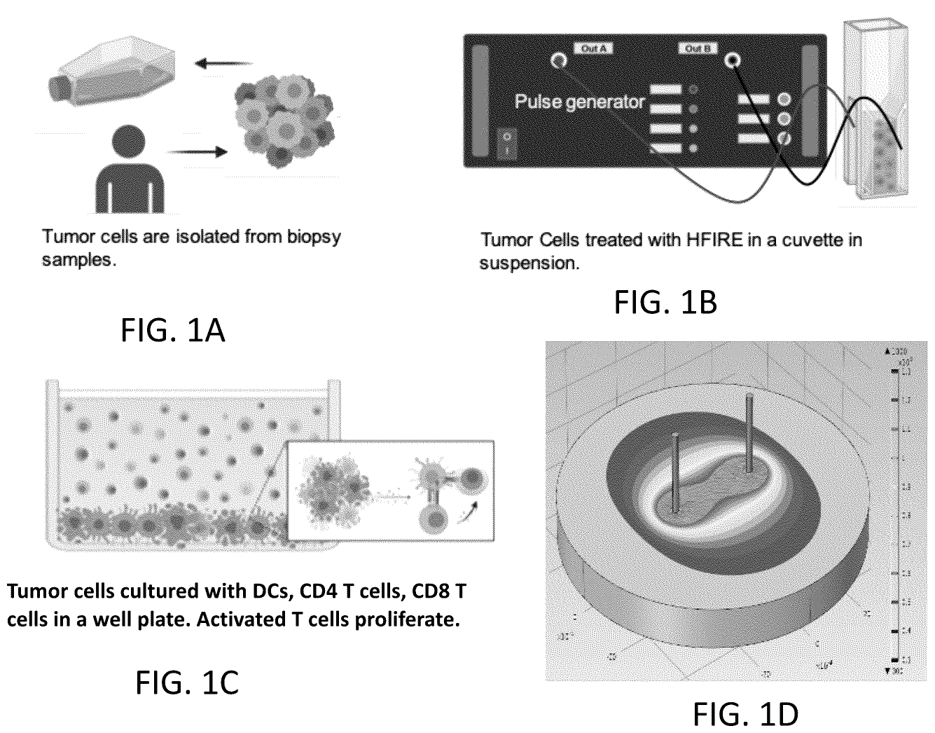

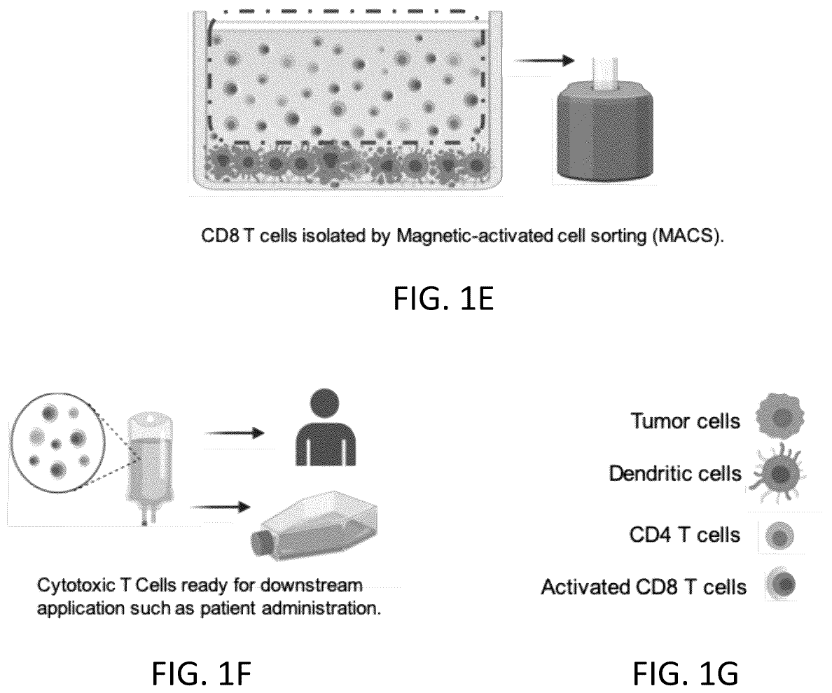

FIGS. 1A-G show a schematic of the coculture platform. As shown in FIG. 1A, tumor cells are isolated from biopsy samples and cultured in vitro or ex vivo. Tumor cells are treated with high voltage pulses in suspension (FIG. 1B), and the treated tumor cells are cocultured with primary dendritic cells, as well as helper and/or cytotoxic T cells (FIG. 1C) in a manner that allows for the activated T cells to proliferate. FIG. 1E shows the separation of the supernatant from the coculture plate through negative selection using magnetic-activated cell sorting, at which point the stimulated cytotoxic CD8+ T cells are ready for downstream application, such as patient administration (FIG. 1F). Cells can also be treated in a custom in vitro tissue microenvironment (TME) to further control the TME during application of the electrical pulses. Electrical pulses can also be delivered to cells in a monolayer, cells cultured in a hydrogel, or other such techniques. In embodiments, multiple cell types can be used. For example, in embodiments a tissue mimic (FIG. 1D) can be used as a substrate for applying the electrical pulses to the cells.

Malignant brain cancer (glioblastoma) cells, U251 (Sigma Aldrich) were cultured in DMEM (ATCC) supplemented with 10% Fetal Bovine Serum (FBS) (Atlanta biologicals) and 1% penicillin-streptomycin (Gibco). Cells were passaged frequently and maintained at 37° C. and 5% CO2 in a cell culture incubator. Normal human dendritic cells from healthy donors (Lonza) were cultured in LGM-3TM media (Lonza) supplemented with 50 ng/ml of IL-4 (interleukin-4) (R&D systems) and 50 ng/ml GM-CSF (R&D systems) for four days. Primary CD4+ and CD8+ T cells (Stemcell Technologies) were cultured in lymphocyte media containing RPMI 1640 (ATCC) supplemented with 12.5 mM HEPES (Life Technologies), 4 mM L-glutamine (Gibco), 100 U/ml penicillin and 100 μm/ml streptomycin (Invitrogen), 50 μM β-mercaptoethanol (Sigma) and 10% normal human serum (Sigma Aldrich). T cells remained in culture overnight prior to stimulation.

U251 cells were trypsinized (Gibco) and resuspended in electroporation buffer (EP buffer) at 1.6×10⁶ cells/ml. Electroporation buffer was composed of RPMI 1640 and a low-conductivity buffer (8.5% sucrose (Fisher Chemical), 0.3% glucose (Acros Organics) and 0.725% RPMI 1640 in water) mixed at the ratio of 1 to 5 respectively. 600 μL of cell suspension, containing approximately 10⁶ cells, was placed in an electroporation cuvette (Harvard Apparatus, 4 mm electrode spacing) and treated with electric pulses of various pulse widths and voltages.

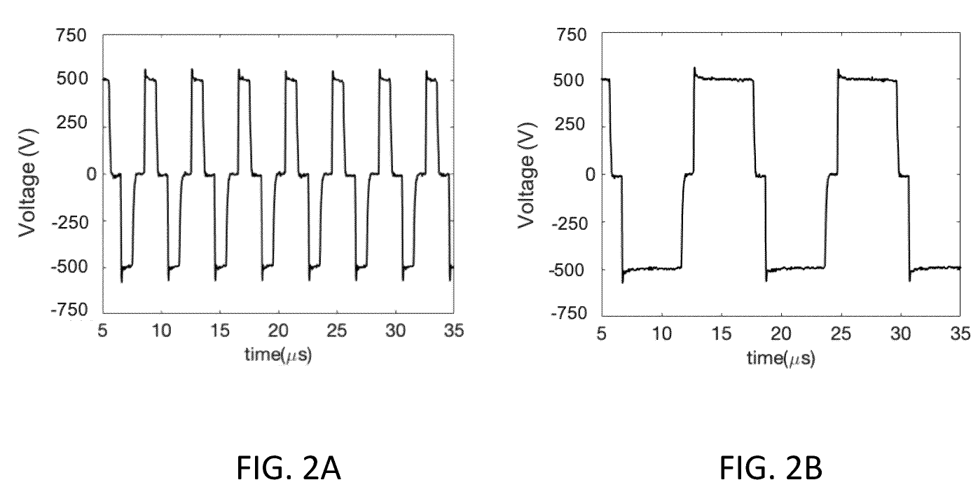

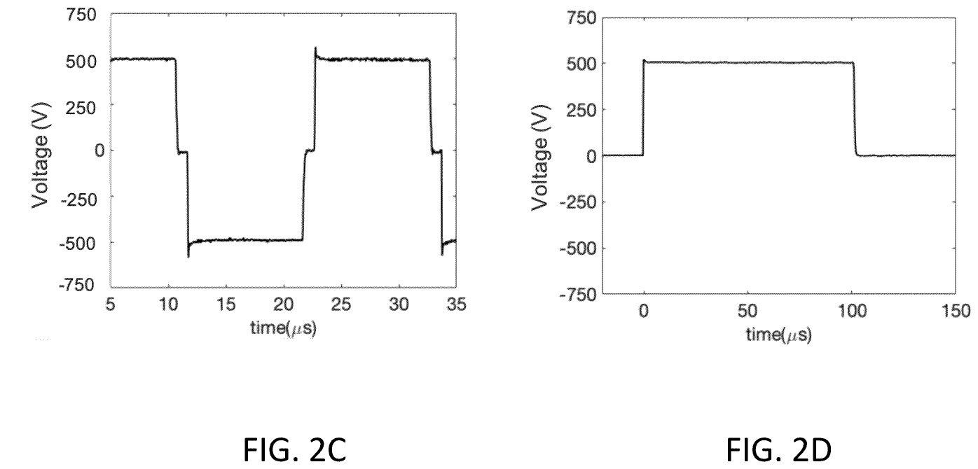

Table 1 presents the HFIRE and IRE pulse conditions utilized in this example. U251 cells were treated with HFIRE waveforms at 500 V and 1000 V (1250, and 2500 V/cm respectively), and with pulse widths of 1, 5, and 10 μs as well as one IRE condition (1250 V/cm, 100 μs pulses). IRE treatments are applied as monopolar pulses, each 100 μs in length, delivered at the frequency of 1 pulse per second. HFIRE treatments are applied as bursts that include bipolar pulses with an inter-pulse delay (1 μs in this example). The number of pulses in each burst is adjusted so that the total voltage application time is 100 μs, the same as the length of a single monopolar IRE pulse. For instance, if the length of a single HFIRE pulse is 5 microseconds, 20 pulses are applied in one burst. HFIRE bursts are also applied at the frequency of 1 burst per second (1 Hz). The HFIRE waveforms are addressed as the positive polarity-delay-negative polarity pulse. For example, a 5-1-5 waveform denotes a 5-μs positive polarity pulse, a 1-μs inter-pulse delay, and a 5-μs negative polarity pulse. FIGS. 2A-D depict example HFIRE (2A-C) and IRE (2D) waveforms recorded from an oscilloscope. It is notable that the IRE pulses normally require lower voltages to achieve the same levels of cell death. Thus, a lower voltage was studied in the IRE treatment group. HFIRE bursts and IRE pulses were delivered at 1 Hz for a total of 100 bursts (HFIRE) or 100 pulses (IRE). HFIRE pulses were delivered as 1-1-1 bipolar pulse bursts (FIG. 2A), 5-1-5 bipolar pulse bursts (FIG. 2B), or 10-1-10 bipolar pulse bursts, at a frequency of one burst per second. The X-X-X convention referred to in this disclosure can mean any one or more of a pulsing protocol of the following formats: pulse ontime-pulse offtime-pulse ontime; positive pulse-delay/offtime-negative pulse; negative pulse-delay/offtime-positive pulse; positive portion of a pulse-delay/offtime-negative portion of pulse, etc. IRE pulses were administered as monopolar pulses at a frequency of one pulse per second. Electroporated U251 cells were centrifuged immediately after treatment, resuspended in lymphocyte media, and plated in a 96 well plate at the density of 10⁵ cells per well.

TABLE 1
List of HFIRE and IRE pulse parameters

| | | Inter- | Applied | | HFIRE/IRE |
|---|---|---|---|---|---|
| | Pulse | pulse | electric | Burst | burst/ |
| | length | delay | field | on-time | pulse |
| Waveform | (μs) | (μs) | (V/cm) | (μs) | number |
| 1-1-1 | 1 | 1 | 1250 | 100 | 100 |
| 5-1-5 | 5 | 1 | 2500 | | |
| 10-1-10 | 10 | 1 | | | |
| IRE | 100 | N/A | 1250 | N/A | 100 |

For comparison, cells were also mechanically lysed using a probe sonicator. Approximately 10⁶ cells were suspended in EP buffer and treated with a 120 W probe sonicator (Fisher scientific) at 20 kHz for 10 seconds for a total of 5 times. Cells were kept on ice to avoid excessive heating. Observing the cells under the microscope following the sonication ensured that the cells had gone through effective membrane lysis.

An untreated control group was also included in this example. 10⁶ cells were suspended in EP buffer and placed in a cuvette for 100 seconds. Cells were then centrifuged and resuspended in lymphocyte media at the same density as the treatment groups.

To ensure HFIRE treatment did not cause thermal damage to the cells, experiments were performed at room temperature and the temperature rise was measured using a fiber optic temperature probe (LumaSense Technologies) for the highest voltage applied at each waveform. For fiber optic temperature measurements, 600 μL of cell suspension was placed in an electroporation cuvette with 4 mm electrode spacing. Fiber optic probes were placed in the samples for temperature reading. IRE or HFIRE pulses were applied at 1250 V/cm and 2500 V/cm respectively. The temperature was measured every 0.5 seconds for a total of 4 minutes.

After HFIRE treatment, the viability of U251 cells was measured through the metabolic assay alamarBlue. The alamarBlue assay measures the fluorometric changes of the dye resazurin to its highly florescent form resorufin as a response to cellular metabolic activity. Treated and untreated (control) U251 cells were plated in a 96 well plate at the density of 3×10⁵ cells per well and maintained in a cell culture incubator overnight. AlamarBlue reagent (Thermo Fisher) was added to each well at a 1:5 ratio of dye to media. Following a two-hour incubation, the fluorescence was measured using a plate reader (Molecular devices) (Ex. 530, Em. 600 nm). The percent viability of the treatment groups was normalized to the untreated control group.

Dendritic cells were lifted from the culture plate using a cell scraper and added to the 96 well plate with treated U251 cells at the density of 10⁵ cells per well. 24 hours later, CD4+ and CD8+ T cells were added to the coculture system each at the density of 5×10⁴ cells per well. CD8+ T cells were stained with CellTrace CFSE (Carboxyfluorescein succinimidyl ester) (Thermo Fisher) before being added to the coculture system. CFSE is a cell-permeant fluorescent dye that is retained in daughter cells upon division due to its covalent bonds with intracellular molecules. The decrease in fluorescent intensity of the dye can be analyzed through flow cytometry and is an indication of cell division. Here, CD8+ T cells were resuspended in PBS (10⁶ cells/ml) and were incubated with 5 μM of CFSE at room temperature for 5 minutes. 5% FBS was added to the cell suspension to avoid toxicity of CFSE. Next, lymphocyte media was added to the cell suspension followed by an extra 5-minute incubation at room temperature. The dye solution was subsequently removed by centrifugation. The coculture plate remained in a cell culture incubator at 5% CO2 for 72 hours. After 72 hours cells were collected from the supernatant, resuspended in PBS and stained with 0.1 μg/ml DAPI (Sigma) to distinguish the dead cells, followed by flow cytometry analysis (Guava simple flow, Millipore).

Samples were analyzed in a capillary-based flow cytometer (Guava simple flow, Millipore). Fully stimulated non-stained cells were used as a control. In addition, undivided cells were stained and analyzed immediately after staining. 50,000 events were acquired for samples from each treatment group. The results were analyzed using the FlowJo software (Version 10.6.1). Scatter plots were gated for live singlet lymphocytes that were positive for CFSE. The CFSE histograms were analyzed with the proliferation module in FlowJo software to find the proliferation index and percent division. All model parameters were fixed and applied to each treatment group.

Samples were collected from coculture wells after 72 hours of incubation. Sandwich ELISA was performed using R&D systems ELISA DuoSet kit for measuring human IL-2 and IFN-γ. The assay was performed following the manufacturer's instructions. Briefly, capture antibodies were incubated in ELISA plates (R&D systems DuoSet) overnight. Samples were added to each well and incubated for 2 hours at room temperature. Next, samples were removed and the wells were incubated with biotinylated detection antibodies for two hours at room temperature. Wells were then incubated with streptavidin HRP for 20 minutes followed by the addition of the substrate solution (1:1 mixture of H₂O₂ and Tetramethylbenzidine). The reaction was stopped by the addition of the stop solution (2N H₂SO₄). The absorbance readings of the plate were measured at 450 nm and corrected by subtracting the absorbance readings at 540 nm. The concentrations of IL-2 and IFN-γ were measured using a standard curve.

The cytotoxicity of the activated CD8+ T cells on U251 cells was evaluated after activation by two HFIRE waveforms (10-1-10 and 5-1-5 at 2500V/cm) as well as an IRE waveform. The results were compared to cytotoxicity of the non-stimulated T cells. The coculture platform was established as explained previously and maintained in the incubator for 72 hours. Mojosort biotinylated antibody cocktail (Biolegend) was used for negative selection of CD8+ T cells. Streptavidin-conjugated magnetic beads were used to bind to the antibody-labeled cells. The antibody-selected cells were then separated and discarded using a magnetic column (Biolegend). Cytotoxicity of CD8+ T cells was measured using CYQUANT LDH assay (Invitrogen). Lactate dehydrogenase (LDH) is an enzyme that indicates damage to the cell plasma membrane. Hence, higher levels of LDH is an indication of higher cytotoxicity. 20,000 untreated U251 cells were seeded in each well of a 96-well plate. 10⁵ isolated CD8+ T cells were added to each well followed by a 24-hour incubation. The cytotoxicity was calculated using Equation 1, as recommended by the manufacturer.

$$Cytotoxicity = \frac{LDH activity of treated cells - spontaneous LDH activity}{Maximum LDH activity - spontaneous LDH activity} \qquad ( 1 )$$

where spontaneous and maximum LDH activity were found from untreated and chemically lysed (following the manufacturer's instructions) U251 cells, respectively.

Each data set was collected from a single batch of human CD4+, CD8+ and dendritic cells from healthy donors. At least three replicates per treatment group were analyzed for the alamarBlue and ELISA assays. For the CFSE assay, two subsamples were analyzed from two independent samples. The LDH assay results were gathered from 5 independent replicates. One-way ANOVA was performed to study the effects of the treatment. Tukey analysis was performed to distinguish the groups that showed statistical significance. P values of P<0.05 were considered statistically significant.

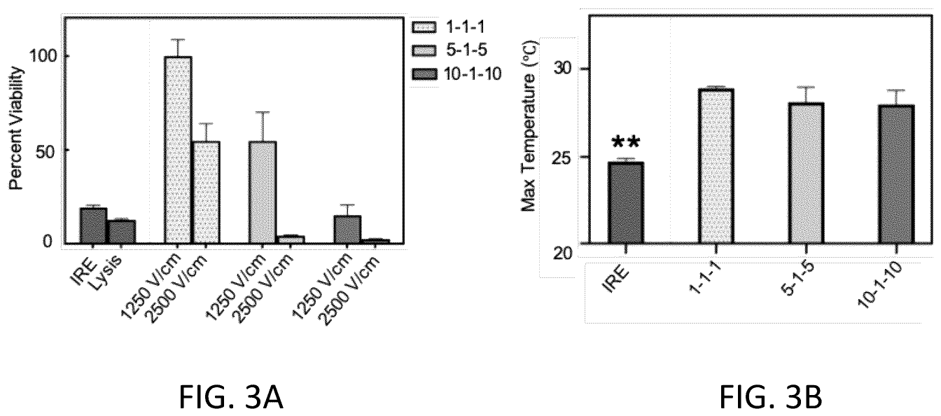

As illustrated in FIGS. 3A-B, cell viability post-HFIRE depends on both the applied voltage and the pulse width. In embodiments, cell death can occur from the plurality of electrical pulses due to any one or more of apoptosis, pyroptosis, necroptosis and/or necrosis. Cell viability and temperature rise after HIRE and IRE treatment were measured. The viability of U251 cells was evaluated using the alamarBlue assay. At 2500 V/cm, all waveforms showed a significant decrease in cell viability comparing to the no-treatment control group. As expected, applying longer pulse widths resulted in lower cell viabilities. Treating cells with 5-1-5 and 10-1-10 waveforms at 2500 V/cm led to nearly complete cell death (FIG. 3A). IRE treatment at 1250 V/cm and mechanical cell lysis were also done to compare with the HFIRE pulses. U251 cells showed comparable loss of viability for IRE and mechanical lysis treatments.

To ensure the viability of treated U251 cells was not affected by the thermal damage associated with Joule heating, the temperature rise was measured for the highest applied voltage across the waveforms. As shown in FIG. 3B, all HFIRE treatments at 2500 V/cm showed moderate temperature increases of ˜5° C. Treating cells with the 1-1-1 waveform caused a slightly greater increase in temperature (no statistical significance). IRE treatment at 1250 V/cm resulted in the lowest increase in temperature of only ˜2° C., due mainly to the lower applied voltage. Since the treatments were performed at room temperature, cell death caused by thermal damage is not expected.

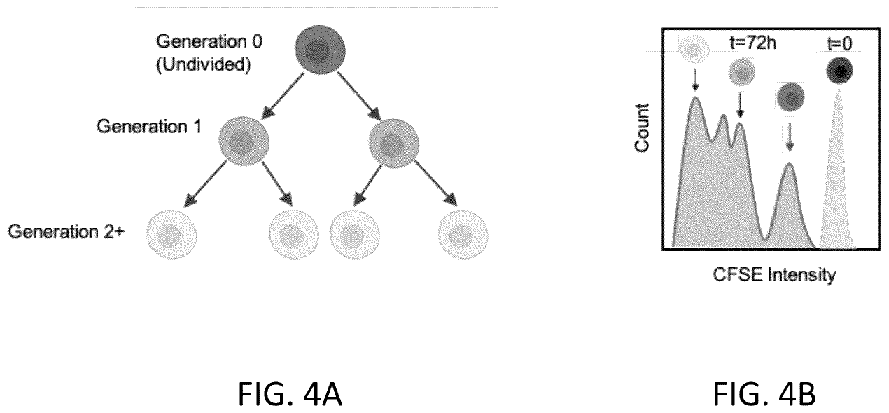

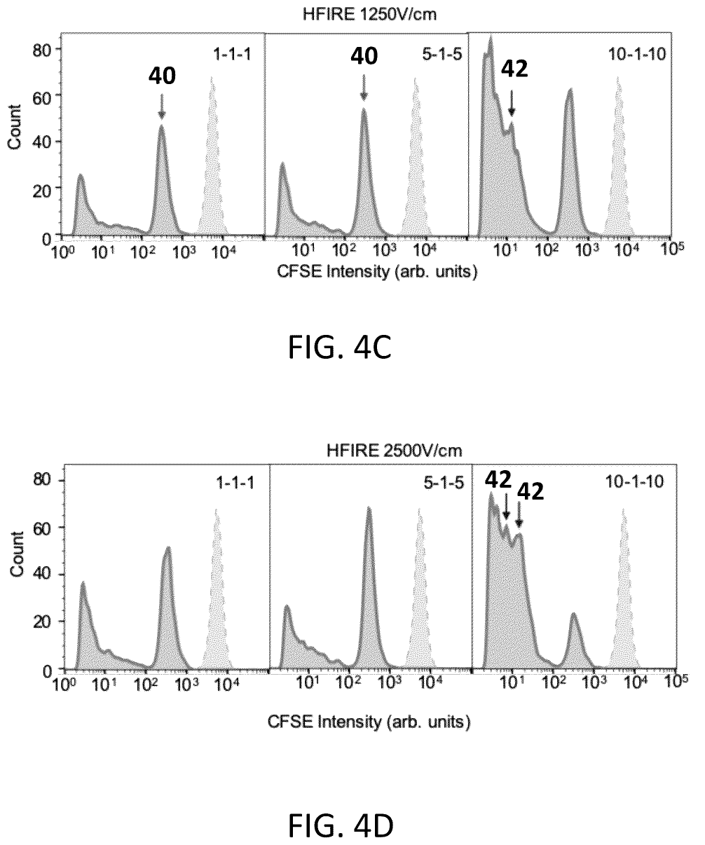

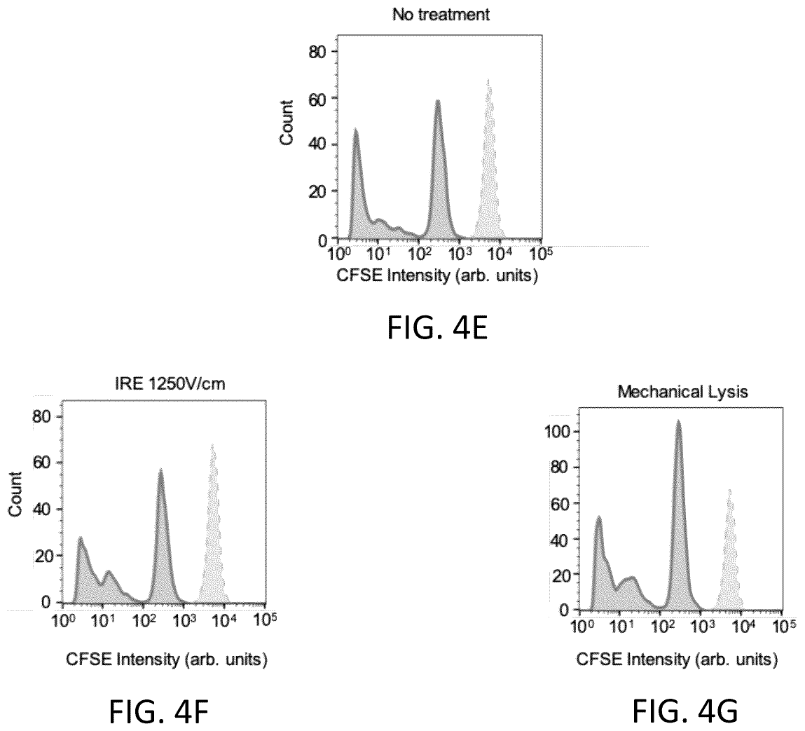

Proliferation of T cells was assessed from CFSE assay results, presented in the histograms in FIGS. 4B-G. As shown in FIGS. 4A-B, the fluorescent intensity of CFSE is expected to decrease in each generation. As a result, separated peaks can be detected in the histogram plots after flow cytometry analysis. Each peak in the histograms represents one generation due to the differences in fluorescence intensity (FIG. 4B). As expected, T cells analyzed immediately after staining with CFSE (t=0), showed high florescent intensity peaks which are overlaid and outlined with dashed lines on the histograms in all treatment groups. The histograms show peaks outlined with solid lines that demonstrate the results of CFSE 72 hours after staining.

The results suggest that maintaining CD8+ T cells in the coculture for 72 hours triggered a significant increase in their proliferation under at least two of the treatment conditions (10-1-10, 2500V/cm and 10-1-10, 1250 V/cm). All other HFIRE treatment groups showed similar trends in their histograms compared to the no treatment control (FIG. 4E). As expected, the peaks associated with the non-proliferative cells, indicated by arrows 40 (FIG. 4C), were shifted to lower fluorescent intensities comparing to the peaks associate with CFSE intensity at t=0. This shift in florescent intensity can be attributed to the loss of dye in undivided cells after 72 hours of incubation. Three representative generation peaks for the 10-1-10 groups are shown by arrows 42 (FIGS. 4C-D). The number of peaks in the histograms represent the number of generations in the proliferative cells.

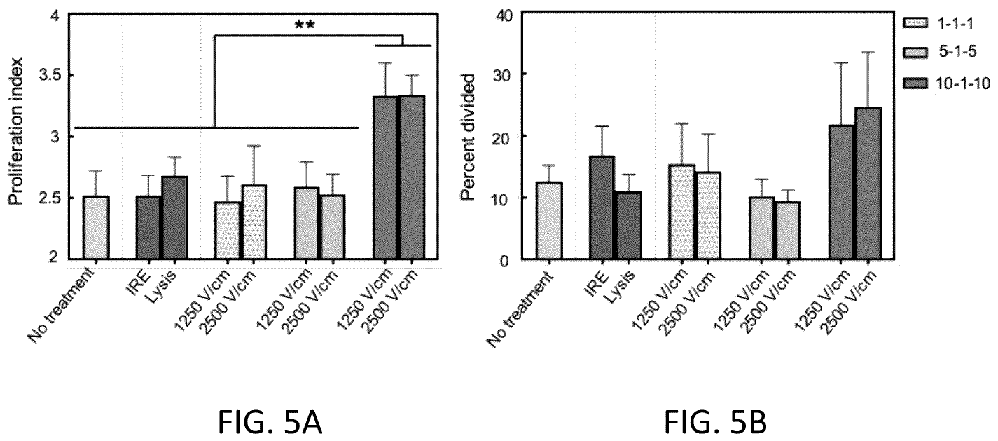

Quantitative analysis was done on the CFSE histograms using the proliferation module of the FlowJo software (Version 10.6.1). FIG. 5A shows the proliferation index and FIG. 5B shows the percentage of division found from the FlowJo proliferation analysis. The proliferation index is calculated as the number of divisions divided by the number of the cells that underwent at least one division. As depicted in the figure, the proliferation index was significantly higher for the 10-1-10 group for both 1250V/cm and 2500V/cm fields. Similar trends were observed for the percentage of divided cells with none of the treatment groups showing a statistically significant difference with the no treatment group. Comparable proliferation index values were found for the two applied fields of all waveforms with no significant difference between the two. The 10-1-10 group showed slightly higher division overall with the highest percentage of cells that divided. Altogether, the results suggest that the 10-1-10 treatment group leads to the highest rates of CTL proliferation.

To further confirm the proliferation of T cells, the concentrations of two important inflammatory cytokines, IL-2 and IFN-γ, were measured in the coculture media. IL-2 is one of the major cytokines that controls the dynamics of CTL activation. Mostly produced by CD4+ helper cells, it is known that IL-2 plays an immunostimulatory role in T cell dynamics by supporting the activation and proliferation of CTLs (Bamford, R. N. et al., The Interleukin (II)-2 Receptor-Beta Chain Is Shared by Il-2 and a Cytokine, Provisionally Designated Il-T, That Stimulates T-Cell Proliferation and the Induction of Lymphokine-Activated Killer-Cells. Proceedings of the National Academy of Sciences of the United States of America 1994, 91 (11), 4940-4944; Gillis, S. et al., Long-term culture of human antigen-specific cytotoxic T-cell lines. J Exp Med 1978, 148 (4), 1093-8; Bachmann, M. F.; Oxenius, A., Interleukin 2: from immunostimulation to immunoregulation and back again. Embo Reports 2007, 8 (12), 1142-1148.). Furthermore, the production of IL-2 can promote differentiation of T cells into effector and memory subsets (Cho, J. H. et al., An intense form of homeostatic proliferation of naive CD8+ cells driven by IL-2. J Exp Med 2007, 204 (8), 1787-801; Kamimura, D.; Bevan, M. J., Naive CD8(+) T cells differentiate into protective memory-like cells after IL-2-anti-IL-2 complex treatment in vivo. Journal of Experimental Medicine 2007, 204 (8), 1803-1812).

Produced by both helper and cytotoxic cells, IFN-γ is known to support proinflammatory pathways in T cell activation (Whitmire, J. K. et al., Interferon-gamma acts directly on CD8+ T cells to increase their abundance during virus infection. J Exp Med 2005, 201 (7), 1053-9). In addition, IFN-γ directly enhances antigen presentation and promotes CTL cytotoxicity (Bhat, P. et al., Interferon-gamma derived from cytotoxic lymphocytes directly enhances their motility and cytotoxicity. Cell Death Dis 2017, 8 (6), e2836; Boehm, U. et al., Cellular responses to interferon-gamma. Annu Rev Immunol 1997, 15, 749-95).

Since the 10-1-10 group showed an increase in CTL proliferation in the CFSE assay, the concentrations of IL-2 and IFN-γ were measured for both applied electric fields. Among the HFIRE waveforms the longer pulse widths corresponded with lower viability of treated tumor cells. Hence, the 5-1-5 group was also included in the measurements for comparison. The concentrations of IL-2 and IFN-γ were also measured for the IRE group.

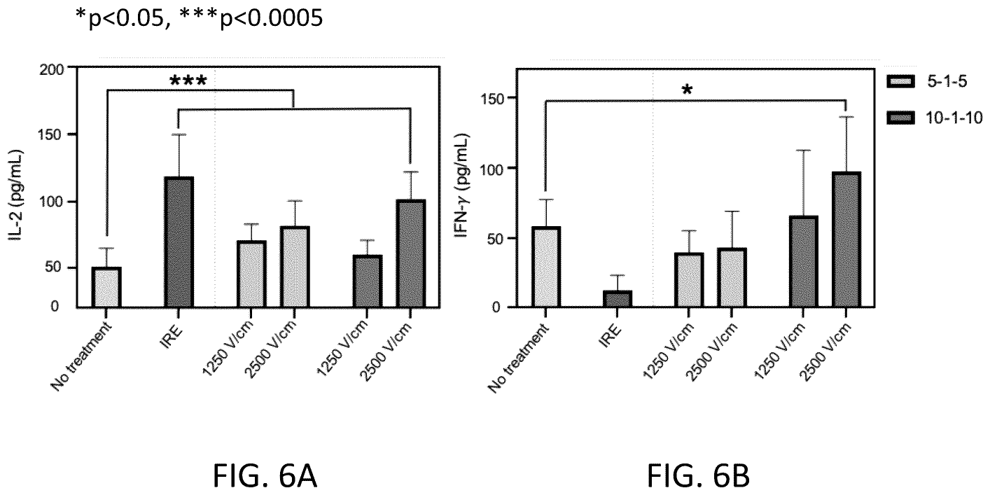

Elevated concentrations of both IL-2 and IFN-γ were detected in the 72-hour coculture media from the 10-1-10 groups as shown in FIGS. 6A-B. For the 5-1-5 group, the concentration of IL-2 was increased while IFN-γ was found to be comparable to the no treatment group. The levels of IL-2 and IFN-γ production were the highest when the higher electric field was applied (2500 V/cm) with 10 μs pulses. The IRE group also showed elevated IL-2 concentration indicating T cell stimulation. However, the concentration of IFN-γ was found to be the lowest for the IRE group. Increased IL-2 and IFN-γ concentrations for 10-1-10 waveforms at 1250 and 2500 V/cm confirm activation and proliferation of T cells in the coculture system for these treatments.

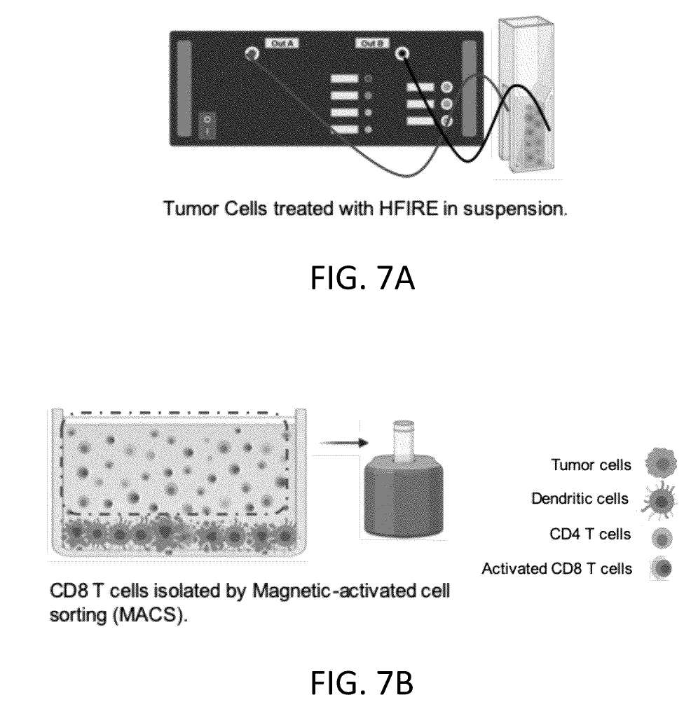

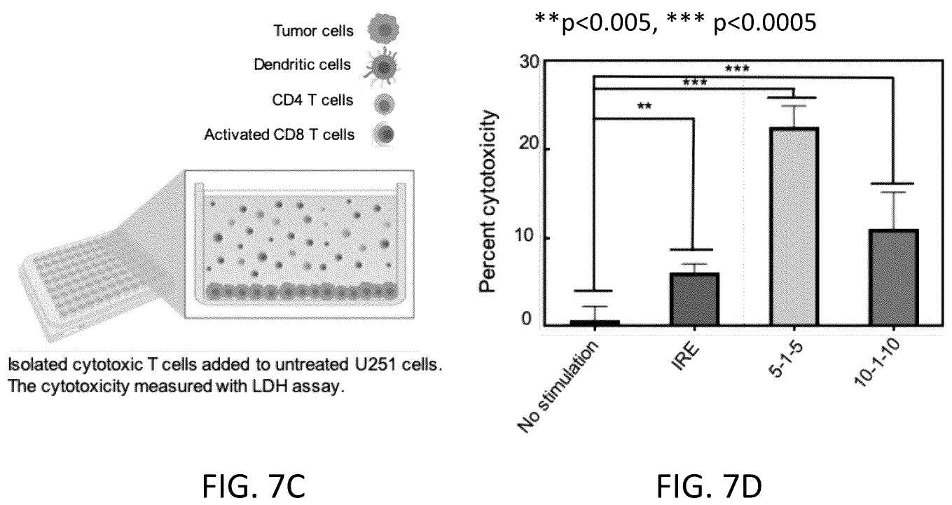

The LDH assay was performed to measure the cytotoxicity of the activated T cells (FIGS. 7A-D). In order to compare the functionality of CTLs activated with different HFIRE pulse schemes against tumor, two longer HFIRE waveforms were chosen at higher applied electric fields (2500 V/cm) as well as the IRE group (1250 V/cm). FIGS. 7A-C show the schematics for the procedure in the cytotoxicity assay. As shown in the schematics, the activated CTLs were isolated through negative selection (FIG. 7B) and added to untreated U251 cells to evaluate their cytotoxicity (FIG. 7C). The results of the cytotoxicity LDH assay indicate that both HFIRE and IRE-activated T cell populations induced higher cytotoxicity on untreated U251 cells comparing to the non-stimulated T cells (FIG. 7D). Increased LDH activity was observed in all treatment groups. Interestingly, the 5-1-5 group showed significantly higher cytotoxicity against U251 cells than all other groups. The 10-1-10 and IRE groups showed comparable cytotoxicity against untreated tumor cells. Based on these findings it can be concluded that HFIRE-stimulated cytotoxic T cells can effectively target untreated tumor cells.

A novel method for stimulating and expanding tumor-activated cytotoxic T cells in vitro or ex vivo using HFIRE pulses has been disclosed herein. Expansion of CTLs is a direct result of their stimulation. Here, it is shown that CTLs undergo division after remaining in the coculture system for 72 hours with HFIRE treated tumor cells, as well as antigen-presenting cells and helper T cells. The inventive platform is designed for the direct activation of cytotoxic T cells through exposure to tumor cells treated with HFIRE pulses. No other commonly-used stimulation techniques, such as antibodies or cytokines, were used here. The present method can also be used for studying the mechanism and dynamics of HFIRE-induced T cell activation, as well as finding the optimum treatment and coculture parameters for production of tumor-activated cytotoxic T cells.

In the example, to stimulate CTLs HFIRE pulse schemes were used with pulse widths of 1-10 μs along with monopolar 100-μs pulses (IRE). The same method can be exploited to study the efficiency of CTL activation for other pulse schemes such as nanosecond pulses. It has been shown that application of nanosecond pulses can result in a T cell-dependent immune response in vivo (Nuccitelli, 2015; Nuccitelli, R. et al., Non-thermal nanoelectroablation of UV-induced murine melanomas stimulates an immune response. Pigment Cell Melanoma Res 2012, 25 (5), 618-29).

A clinically relevant protocol for the production of tumor activated T cells might use patient-derived autologous immune cells. Due to limitations of availability of autologous cells, human CD4+, CD8+ cells were used, and dendritic cells from different healthy donors. It is important to note that although some variability in T cell response can be expected from donor to donor, the overall trend of the result is expected to be comparable to the work presented herein.

As expected, applying higher electric field strengths and longer pulses resulted in overall more effective cell death in U251 cells. It is anticipated that the presence of dead cells, as well as higher levels of damage associated molecular patterns (DAMPs) might lead to more effective stimulation of CTL proliferation. However, considering the lower extent of T cell proliferation in response to the 5-1-5 waveform at 2500V/cm, it can be concluded that the cell viability cannot be the only determining factor in the T cell activation dynamics. It is indeed important to note that applying different pulse schemes may lead to different modes of cell death and consequently different levels of antigen presentation.

Among the various tested waveforms and voltages, the results showed that the 10-1-10 waveforms at 2500V/cm and 1250 V/cm induced the highest rates of T cell proliferation. No significant difference was observed in proliferation of CTLs between the two applied fields. However, when the concentration of IL-2 and IFN-γ were measured, the higher applied fields showed higher production of both cytokines, suggesting more effective activation of CTLs. Both IL-2 and IFN-γ play immunostimulatory roles in T cell activation. It is also notable that both IL-2 and IFN-γ can induce immunosuppressive effects on T cell dynamics. IL-2 is known to regulate the immune response by controlling the proliferation of regulatory T cells (Tregs) (Laurence, A. et al., Interleukin-2 signaling via STATS constrains T helper 17 cell generation. Immunity 2007, 26 (3), 371-81). IFN-γ plays a role in T cell homeostasis by contraction of antigen-specific cells (Tewari, K. et al., Role of direct effects of IFN-gamma on T cells in the regulation of CD8 T cell homeostasis. J Immunol 2007, 179 (4), 2115-25). Considering the complex role of both cytokines and more specifically, their immunosuppressive roles on antigen-specific effector cells, the lower efficiency of the 10-1-10 group in the cytotoxicity assay can be better understood. While the 10-1-10 treatment groups showed increased proliferation of CD8+ T cells in the CFSE assay, the LDH assay shows higher effector function for the 5-1-5 treatment group at 2500V/cm. Overstimulation of CD8+ T cells in the 10-1-10 group might have resulted in the CD8+ cells losing some of their cytotoxicity effects. Similar effects can be imagined for the IRE group. In fact, the LDH cytotoxicity assay results show the reverse trend of the IL-2 secretion, which strengthens the hypothesis that the higher concentrations of IL-2 can induce a regulatory role on effector function of T cells after 72 hours of stimulation. To avoid the immunosuppressive effects resulting from overstimulation, methods such as blockade of immunosuppressive pathways can be utilized. Hence, techniques such as the addition of anti-PD1 might be effective for increasing the efficiency of T cell activation. In fact, Zhao et al. showed increased efficiency of IRE treatment when combined with anti-PD1 treatments in a pancreatic mouse model (Zhao, 2019). The level of IFN-γ was found to be significantly low for the IRE group. Since the CD8+ T cells play an important role in secretion of IFN-γ, it was hypothesized that the low proliferation of CD8+ T cells in the IRE group might have resulted in lower levels of IFN-γ.

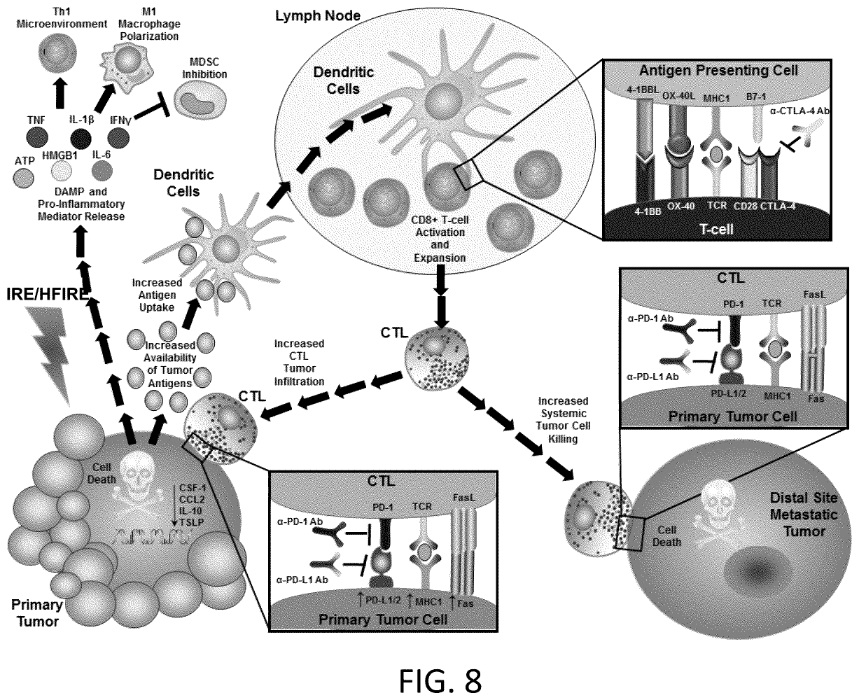

It is important to keep in mind that T cell activation is a complex process with many factors playing into its dynamics. FIG. 8 shows an exemplary process of T cell activation in vivo after administration of electroporation. In embodiments, electroporation, such as HFIRE or IRE, cause the release of danger signals or DAMPs, such as HMGB1. DAMP signaling then stimulates a systemic response for targeting distant lesions. T cells become activated to cytotoxic T cells and travel systemically to recognize and destroy tumors. Non-thermal electroporation allows for antigen release in native form, which in turn provides for a more robust systemic response when compared to thermal treatment. To fully understand how T cell activation occurs after electroporation, the role of other pro- and anti-inflammatory cytokines such as IL-10, IL-4, IL-12, TNF-α could be studied.

The current protocol explains a potential method for expansion of T cells. However, as observed with the 10-1-10 group, the effector function of CTLs seems to be dampened by overstimulation. To overcome this issue, or in other words, to capture the CTLs in their most optimum functionality and proliferation capacity, the timing and the extent of stimulation can be optimized. For instance, the ratio of DCs to T cells could be reduced in the coculture wells. Alternatively, CTLs could be isolated from the coculture after shorter incubation times in the coculture systems.

The extent of proliferation for different subtypes of cytotoxic T cells was not studied here. More detailed analysis of various T cell subtypes such as deep sequencing of T cell receptor (TCR) might provide helpful insights regarding the efficiency of the tumor activated clones in CTLs (Poschke, I. C. et al., The Outcome of Ex Vivo TIL Expansion Is Highly Influenced by Spatial Heterogeneity of the Tumor T-Cell Repertoire and Differences in Intrinsic In Vitro Growth Capacity between T-Cell Clones. Clin Cancer Res 2020, 26 (16), 4289-4301).

The results suggest that higher voltage and longer HFIRE pulses lead to greater enhancements in CD8+ T Cell proliferation. However, a variety of different HFIRE parameters can be used with this method, such as different voltages, numbers of pulses, inter-pulse delays, intra-pulse delays, etc.

The supernatant according to an embodiment of the coculture system contained a mixture of CD4+ and CD8+ T cells, along with debris from treated tumor cells. Cytotoxic CD8+ T cells could be isolated from the rest of the suspension using various methods such as MACS, FACS or microfluidic platforms as described previously (Alinezhadbalalami, N. et al., The feasibility of using dielectrophoresis for isolation of glioblastoma subpopulations with increased stemness. Electrophoresis 2019, 40 (18-19), 2592-2600; Shafiee, H.; Sano, M. B. et al., Selective isolation of live/dead cells using contactless dielectrophoresis (cDEP). Lab Chip 2010, 10 (4), 438-445). The platform can also be exploited for investigating the dynamics of HFIRE-mediated activation of CTLs. For instance, the optimum pulse parameters can be found through the co-culture system to achieve the best stimulation response. In other embodiments of the invention, antibodies or cytokines can be combined with the method to generate synergistic effects. Additionally, the activated T cells can also be used for engineering different types of CAR T cells to enable specific tumor targeting and selectivity.

The present invention demonstrates lymphocyte expansion and activation in vitro or ex vivo using HFIRE. Here, the inventive coculture platform induces CD8+ T cell activation and proliferation by exposure of T cells to HFIRE-treated tumor cells. HFIRE treatment with high voltages and with waveforms with longer pulse durations (5-10 μs) induce significant tumor cell death, and can enhance CD8+ T cell proliferation and activation. Activated CD8+ T cells were shown to be cytotoxic to untreated tumor cells. Embodiments of the method of T cell expansion provide an alternative to conventional techniques, and have the unique advantage of not requiring antibodies or cytokines.

1. A method comprising:
subjecting one or more patient cell to a plurality of electrical pulses, wherein the subjecting is performed in vitro or in vivo or ex vivo; and
performing one or more of:
exposing the one or more patient cells to peripheral blood mononuclear cells (PBMCs), tumor infiltrating lymphocytes (TILs), or dendritic cells;
exposing the one or more patient cells to CD4+ helper T cells and/or CD8+ cytotoxic T cells (CTLs);
generating, activating and/or providing for expansion of one or more tumor-reactive T cells and/or cytokines;
separating activated tumor-reactive T cells and/or activated cytokines out; and
inoculating or exposing a patient with/to the activated tumor-reactive T cells and/or activated cytokines.

2. A method for producing cytotoxic T lymphocytes, comprising:
positioning one or more electrodes near a target area containing cells to be treated;
applying a plurality of electrical pulses to the target area through the electrodes;
exposing treated cells to dendritic cells; and
exposing the treated cells to one or more types of T lymphocytes.

3. (canceled)

4. The method of claim 1, wherein no cytokines or antibodies are added.

5. The method of claim 1, wherein the plurality of electrical pulses are administered at a voltage in the range of above 0 V to 10,000 V.

6. The method of claim 1, wherein pulses of the plurality of electrical pulses have a pulse width in the range of from about 1 picosecond to 50 microseconds.

7. The method of claim 1, wherein the plurality of electrical pulses comprise HFIRE pulses administered according to a positive pulse-delay-negative pulse protocol or a negative pulse-delay-positive pulse protocol.

8. The method of claim 1, wherein the plurality of electrical pulses has a frequency in the range of above 0 Hz to 100 MHz.

9-11. (canceled)

12. The method of claim 1, wherein the plurality of electrical pulses has a pulsing scheme with one or more intra- or inter-pulse delays.

13. The method of claim 1, wherein the plurality of electrical pulses has a pulsing scheme conforming to the following formula:
(i) administering a pulse with a first polarity and a pulse duration of less than 10 microseconds,
(ii) administering a delay with a duration of up to 20 microseconds,
(iii) administering a pulse with a second polarity, which is the same or a different as the first polarity, and a pulse duration of less than 10 microseconds,
(iv) administering a delay of up to 1 second, and
(v) repeating the administering of (i)-(iv) a desired number of times.

14. (canceled)

15. A method comprising:
applying a plurality of electrical pulses to a cell sample in vitro or ex vivo;
culturing the cell sample with dendritic cells;
exposing the cell sample and the dendritic cells to helper T cells and/or cytotoxic T cells, such that the cytotoxic T cells proliferate; and
isolating the cytotoxic T cells from the cell sample.

16. (canceled)

17. The method of claim 15, wherein the helper T cells are CD4+ helper T cells and/or the cytotoxic T cells are CD8+ cytotoxic T cells.

18. The method of claim 17, wherein the CD4+ helper T cells and/or the CD8+ cytotoxic T cells are isolated from peripheral blood mononuclear cells or are isolated from an organ.

19. The method of claim 17, further comprising administering the isolated CD8+ cytotoxic T cells to a patient.

20. The method of claim 17, further comprising exposing the isolated CD8+ cytotoxic T cells to a second cell sample in vitro or ex vivo, wherein the second cell sample is a cell sample from an individual who is different from which the first cell sample was obtained.

21-22. (canceled)

23. The method of claim 17, further comprising exposing the cell sample and human dendritic cells to cytokines and/or antibodies.

24. (canceled)

25. The method of claim 15, wherein:
the cell sample is obtained and isolated from a first subject.

26. (canceled)

27. The method of claim 25, further comprising administering the isolated cytotoxic T cells into a second cell sample or into a second subject, which second cell sample or second subject is the same or a different cell sample or subject from the first subject.

28-32. (canceled)

33. A method comprising:
applying a plurality of electrical pulses to one or more cells of a subject in vivo;
isolating one or more of the cells of the subject;
culturing the one or more cells with dendritic cells;
exposing the one or more cells and dendritic cells to helper T cells and/or cytotoxic T cells; and
isolating the cytotoxic T cells.

34. (canceled)

35. A method comprising:
applying a plurality of electrical pulses to a cell sample in vitro or ex vivo;
culturing the cell sample with human dendritic cells.

36-39. (canceled)

40. The method of claim 15, wherein the cytotoxic T cells are isolated from the cell sample by way of magnetic-activated cell sorting.

41-49. (canceled)
